(19) United States
(12) Patent Application Publication
(10) Pub. No.: US 2010/0120736 A1
(43) Pub. Date: May 13, 2010

(54) Mineralocorticoid Receptor Antagonists

(75) Inventors: Bernardus Wijnand Machijs Marie Peeters, Oss (NL); Marinus Bernard Groen, Oss (NL); Ralf Plate, Oss (NL)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. ORGANON

(21) Appl. No.: 12/593,746

(22) PCT Filed: Mar. 29, 2007
(86) PCT No.: PCT/GB07/01141

§ 371 Date: Jan. 15, 2010

(51) Int. Cl. A61K 31/57; C07J 7/00; A61P 43/00

(52) U.S. Cl. 514/177; 552/608

(57) ABSTRACT

Compounds of the formula (I) are provided having a steroid skeleton and substitution characteristics in the A and B rings of the steroid skeleton effective for mineralocorticoid receptor antagonism, and rings C and D of the steroid skeleton having substituents thereon according to formula (I), wherein R¹ is —OH or ═O; R² is (C₁₋₃)alkyl or (C₂₋₃)alkenyl; R³ is selected from formulas (IIa), (IIb), (IIc). These compounds are useful in the treatment of inter alia aldosteronism, hypokalemia, hypertension, congestive heart failure, heart fibrosis, renal failure and restenosis.

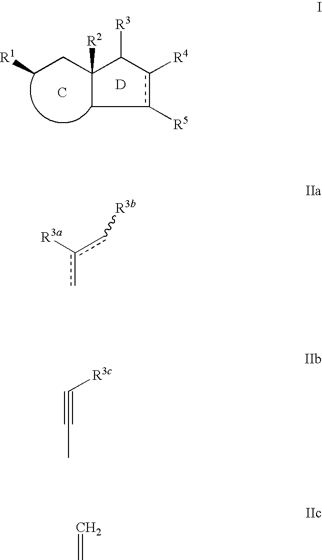

The current invention relates to novel steroid compounds that are mineralocorticoid receptor antagonists and have potential for use in conditions related to the mineralocorticoid receptor.

Aldosterone is involved in the regulation of the fluid and mineral balance in an organism through stimulation of mineralocorticoid receptors; mineralocorticoid receptor antagonists, such as those of the current invention are useful in the treatment of aldosterone imbalance for example in aldosteronism. Aldosterone antagonists promote the elimination of water and sodium, whilst sparing the elimination of potassium and are therefore useful as diuretics, in the prevention and treatment of cardiac dysfunction, the reduction of fluid burden, for example in the treatment edema, and in hypertension and associated conditions.

Treatable conditions also include coronary and vascular fibrosis, for example myocardial fibrosis and cardiac hypertrophy, particularly in left ventricular hypertrophy, heart failure, coronary heart disease, the prevention of damage caused post myocardial infarction, the prevention of myocardial infarct, renal disease, particularly associated with hypertension and diabetes, such as nephropathy, nephrotic syndrome, retinopathy and neuropathy, hepatic cirrhosis, hypokalemia, metabolic syndrome, atherosclerosis, restenosis, cerebrovascular disease, stroke, obesity, endothelial dysfunction, precocious puberty (particularly in boys), polycystic ovary syndrome and premenstrual syndrome.

A number of aldosterone antagonists are known including for example spironolactone, eplerenone, drospirenone, mexrenone and canrenone. The aldosterone antagonists spironolactone and canrenone, have a spirolactone group at the C-17 position of the steroid skeleton (Cella et al., J. Org. Chem. 24, 743; 1959).

Both spironolactone and canrenone suffer from a number of deficiencies, mostly due to their progestational and anti-androgenic activity. In some cases, in particular with high doses, this results in menstrual irregularities and breast tenderness in women and in gynecomastia, loss of libido and even impotence in men (Martindale, The Extra Pharmacopeia, 31st ed., 946-948). Also, the potency of spironolactone is low and a high daily dose (25 mg or more) is required for efficacy. In recent years the therapeutic scope of aldosterone antagonists has broadened considerably, most notably in the management of congestive heart failure where its efficacy appears to be dramatic. Consequently the need for aldosterone antagonists has enlarged considerably. Not surprisingly, many attempts have been made to find improved aldosterone antagonists. Remarkably little improvements were obtained, however. Since the discovery of canrenone and spironolactone in the late 50's hundreds of compounds have been reported by various research groups and over 20 compounds have been tested clinically. In spite of favourable pharmacological properties in animal tests none of these compounds made it to use in medical practice. The reasons given are i.a, disappointing potency, hepatotoxicity (e.g. mexrenone, SC 25951), unfavourable pharmacokinetics, unfavourable side effect profile, etc. In general in this class of compounds there appears to be a larger than usual discrepancy between animal (rodents, dog) and human pharmacology. An interesting case is spirolenone, which was selected as a selective aldosterone antagonist without significant progestational activity (rat, rabbit). In the clinic this compound turned out to have a mixed progestagenic/aldosterone antagonistic profile, due to efficient metabolism to the 1,2-dihydro derivative. This metabolite is now available on the market as an OC with diuretic properties (drospirenone, Yasmin®)

In the 1980s it was found that introduction of a 9,11-epoxy group into known aldosterone antagonists reduced the progestational and anti-androgenic activity, usually with retention of the aldosterone antagonistic activity (J. Grob et al., Hely. Chim. Acta, 80, 566-505; 1997); M. de Gasparo et al., J. Pharmacol. Exp. Ther., 240, 650-656; 1987). One such compound, eplerenone (Inspra®), has been approved by the FDA for treatment of hypertension (Nature Reviews, Drug Discovery 2, 177-178; 2003). It is worthy of note that almost all research on aldosterone antagonists has relied heavily on the spirolactone lead structure. This may underlie the lack of progress in finding better aldosterone antagonists.

U.S. Pat. No. 5,120,724 discloses steroidal compounds which are inhibitors of aldosterone synthesis, U.S. Pat. No. 2,840,573 discloses 18-oxygenated allopregnanes which are useful in inhibiting salt retention caused by administration of adrenocortical hormones. Compounds, having long carbonyl chains in position 17 of the steroid skeleton are described in Evans et al J. Chem. Soc 1529; 1958; Schneider and Haeffner, J. Chromatography 70, 194-198; 1972; and Schneider Tetrahedron 28, 2717; 1972. Compounds with hydroxyl substitution at position 11 but being in α-configuration, are mentioned in U.S. Pat. No. 4,013,688, Cimino et al (1979), Experimentia 35, 298-299 and U.S. Pat. No. 2,752,369. U.S. Pat. No. 4,180,570 discloses 17β-hydroxy-4-androsten-3-ones which have aldosterone antagonist effects.

It has now been found that such compounds of the formula I with short chain substituents at C17 and 11β-OH substitution, or esters or ethers thereof are effective as mineralocorticoid receptor antagonists. Thus, in a first aspect, the present invention provides mineralocorticoid receptor antagonists having a steroid skeleton and substitution characteristics in the A and B rings of the steroid skeleton effective for mineralocorticoid receptor antagonism, and rings C and D of the steroid skeleton and substituents thereon are according to formula I

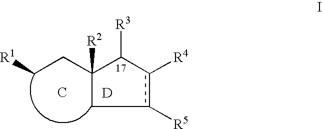

wherein:

R¹ is —OH or =0;

R² is (C₁₋₃)alkyl or (C₂₋₃)alkenyl;

R³ is selected from:

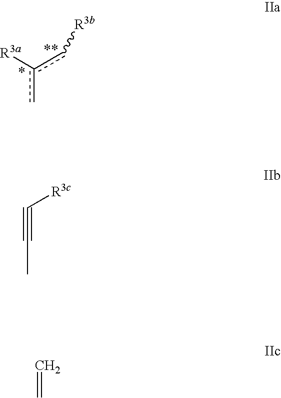

Wherein the lowermost carbon is carbon 17 of the D ring
R^{3a} is H, halogen, monocyclic aryl or is (C₁₋₅)alkyl optionally substituted with hydroxy, halogen, (C₁₋₆)alkoxy or (C₁₋₆)acyloxy;
R^{3b} is H, (C₁₋₃)alkyl or halogen; and
R^{3b} is H, (C₁₋₆)alkyl, (C₂₋₆)alkenyl or (C₂₋₆)alkynyl;

R⁴ is H or (C₁₋₆)alkyl;

R⁵ is H or R⁴ and R⁵ taken together are —CH₂— as part of a 15,16-cyclopropa group and the double bond between R⁴ and R⁵ is absent;

[symbol] is independently in each case either a single bond or a double bond but is a single bond when part of a cyclopropa group.

or a pharmaceutically acceptable ester or ether thereof.

The compounds (11β)-11-hydroxy-pregn-4-en-3-one, (11β-20S)-11,21-dihydroxy-20-methylpregn-4-en-3-one and (11β-20S)-11,21-dihydroxy-20-methyl-pregn-1,4-dien-3-one, are mentioned as synthetic intermediates in, respectively, Vandenhewel (1975); J. Chromatography Vol. 103; pp. 113-134; Petzold et al (1980), DE2839033 and Undisz et al (1992); J Steroid Biochem. Molec. Biol. Vol. 43; pp. 543-547 and are excluded from claims to compounds per se.

It is understood that a double bond between C-17 and the carbon atom denoted “*” cannot co-exist with a double bond between the carbon atom denoted “*” and the carbon atom denoted “**”

If a double bond connects “*” and “**” then it may have a Z or E configuration resulting in a cis or trans relationship between R^{3a} and R^{3b}.

In a preferred embodiment, compounds of the formula I are of the formula III

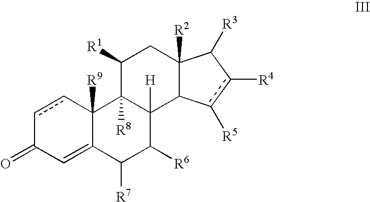

wherein R¹ to R⁵ have the definitions above and:

R⁶ is H, —CN, (C₁₋₆)alkyl, carboxyl(C₁₋₄)alkyl, carboxyl, —C(═O)O(C₁₋₄alkyl (C₁₋₅)alkylthio, or (C₁₋₅)acylthio;

R⁷ is H or halogen, or R⁶ and R⁷ taken together are a —CH₂— group as part of a 6,7 cyclopropa group or, taken together, R⁶ and R⁷ form the second bond of a double bond;

R⁸ is H or a halogen atom, or, taken together, R¹ and R⁸ form the second bond of a double bond;

R⁹ is H or (C₁₋₄)alkyl; and

[symbol] is in each case, independently, either a single bond or a double bond but is a single bond when part of a cyclopropa group,

or a pharmaceutically acceptable ester or ether thereof.

In a further preferred embodiment compounds of the formula I are of the formula IV

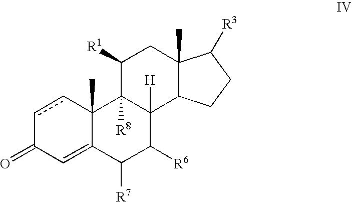

in which the definitions of substituents are as above. Preferably they are compounds in which:

R⁶ is H

R⁷ is H, halogen in the β-configuration, or R⁶ and R⁷ combine as —CH₂— as part of a β-cyclopropa group or, taken together, R⁶ and R⁷ form the second bond of a double bond;

In the above embodiments:

R¹ is preferably —OH;

R² is preferably methyl, ethyl or vinyl; more preferably it is methyl or ethyl and most preferably it is methyl.

R^{3a} is preferably H, halogen, or (C₁₋₅)alkyl optionally substituted with halogen, (C₁₋₆)alkoxy or (C₁₋₆)acyloxy; more preferably R^{3a} is H, halogen, or is methyl or ethyl optionally substituted with halogen, methoxy or (C₁₋₃)acyloxy; more preferably R^{3a} is H, halogen, methyl or ethyl; most preferably it is methyl or ethyl and most preferably methyl.

R^{3b} is preferably H or methyl; and most preferably H.

R^{3c} is preferably H or (C₁₋₆)alkyl; more preferably H or methyl; and most preferably H.

R⁴ is preferably H, ethyl or methyl; more preferably H or methyl and most preferably H.

R⁵ is H or R⁴ and R⁵ taken together are —CH₂— as part of a 14,15 cyclopropa group;

R⁶ is preferably H, —CN, (C₁₋₄)alkyl, carboxyl, —C(═O)OCH₃, (C₁₋₅)acylthio; more preferably R⁶ is H, methyl, ethyl, propyl, carboxyl, —C(═O)OCH₃ or —S(C═O)CH₃, more preferably R⁶ is H, methyl or —S(C═O)CH₃ and most preferably is H or methyl.

R⁸ is preferably H or halogen and is most preferably H.

R⁹ is preferably (C₁₋₄) alkyl, more preferably methyl or ethyl and is most preferably methyl.

Preferably, when R³ is attached to the D ring of the steroid by a single bond, it is in the β configuration. That is to say the D ring may be represented as:

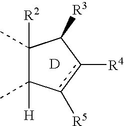

R³ is preferably of the formula IIa or IIb; most preferably is of the formula IIa

Where R³ is of the formula IIa, and a double bond exists between the carbon indicated by “*” and the carbon indicated by “**”, then it is preferred that R^{3a} and R^{3b} are in the cis configuration. That is to say as illustrated in formula V:

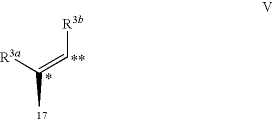

Further preferred embodiments are those in which R^{3a} is H.

Further preferred embodiments are those in which R³ is selected from the group consisting of:

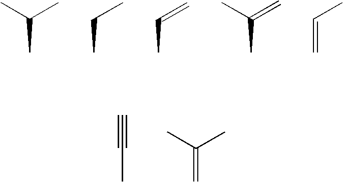

in which the lower most Carbon is carbon 17 of the D ring.

Further preferred embodiments are those in which R³ is selected from the group consisting of:

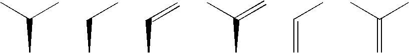

in which the lower most Carbon is carbon 17 of the D ring.

Further preferred embodiments are those in which R³ is selected from the group consisting of:

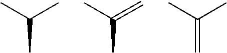

in which the lower most Carbon is carbon 17 of the D ring.

Further preferred compounds of the above embodiments are those in which R⁴ is alkyl; preferably methyl, ethyl or propyl; more preferably methyl or ethyl and most preferably methyl.

Further preferred compounds of the above embodiments are those in which R⁶ is H, (C₁₋₆)alkyl, carboxyl(C₁₋₄) alkyl, (C₁₋₅)alkylthio, or (C₁₋₅)acylthio

further preferred compounds of the above embodiments are those in which R⁶ is H and R⁷ is H, halogen in the n-configuration, or R⁶ and R⁷ combine as —CH₂— as part of a β-cyclopropa group.

Further preferred compounds of the above embodiments are those in which R⁶ and R⁷ taken together do not form a double bond, that is to say, the steroid group is devoid of a 6,7 double bond.

Further preferred compounds of the above embodiments are those in which R⁶ is H and R⁷ is halogen in the β configuration.

Further preferred compounds of the above embodiments are those in which R⁶ is —SC(═O)CH₃

Further preferred compounds of the above embodiments are those in which R⁶ is methyl or ethyl, particularly methyl

Further preferred compounds of the above embodiments are those in which R⁶ is H

Further preferred compounds of the above embodiments are those in which R¹ and R⁸ do not form a double bond when taken together.

Further preferred compounds of the above embodiments are those in which the (C₁₋₅)alkyl group of R^{3a} is not substituted by —OH, (C₁₋₆)alkoxy or (C₁₋₆)acyloxy and more particularly wherein the (C₁₋₅)alkyl group of R^{3a} is unsubstituted.

Where any non cyclic substituent is said to comprise one to six carbon atoms (e.g. (C₁₋₆)alkyl, (C₁₋₆) alkoxy etc.) then it is preferred that it comprises one to three carbons, more preferred that it comprises two carbons and particularly preferred that it comprises one carbon atom. Where a non cyclic unsaturated substituent is said to comprise two to six carbon atoms it is preferred that it comprises 2 or 3 carbon atoms and more preferred that it comprises 2 carbon atoms.

Alkyl is a branched or unbranched alkyl group, for example methyl, ethyl, propyl, isopropyl, iso-butyl, sec-butyl, tent-butyl, hexyl, octyl, capryl, or lauryl.

Halogen is preferably chlorine or fluorine, most preferably fluorine.

The term “monocyclic aryl” refers to a monocyclic aromatic or hetero aromatic ring. In the case of a hetero aromatic ring it may contain up to 2 (preferably one) heteroatoms independently selected from O, S and N. Preferably the ring is either a phenyl or a pyridyl ring, most preferably phenyl.

Preferred ester and ether compounds of the invention are carboxylic acid esters—particularly alkyl (for example C₁₋₆) carboxylic acid esters—or alkyl (for example C₁₋₆) ethers of one or more available hydroxyl groups—particularly of the 11-hydroxy group where present—and more preferred compounds are (C₂₋₆) carboxylic acid esters, such as esters of ethanoic, propanoic or butanoic acid (including iso, sec or tent-butanoic acid), or (C₁₋₄)alkyl ethers, such as methoxy or ethoxy. For example, R¹, in addition to those options cited above, may be —OMe, —OC₂H₅, —OC₃H₇ or —OC₄H₉, or may be —O(C═O)H, —O(C═O)Me, —O(C═O)C₂H₅, —O(C═O)C₃H₇ or —O(C═O)C₄H₇.

Where compounds of the invention are able to form salts, for example where R⁷ is a group comprising a carboxyl moiety, pharmaceutically acceptable salts of these compounds are included in the scope of the invention.

For the avoidance of doubt the following numbering scheme has been used in the this text:

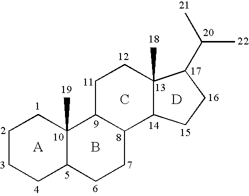

The following compounds exemplify those of formula 1:

TABLE 1

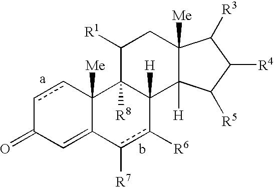

| No. | R¹ | R³ | R⁴ | R⁵ | R⁶ | R⁷ | R⁸ | a | b |
|---|---|---|---|---|---|---|---|---|---|
| 1 | —OH | 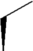 | H | H | H | H | H | S | S |
| 3 | —OH | 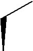 | H | H | H | H | H | D | S |
| 4 | —OH | 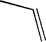 | H | H | H | H | H | S | S |
| 6 | —OH | 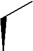 | H | H | Me | H | H | D | S |
| 8 | —OH | 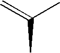 | H | H | H | H | H | S | S |
| 12 | —OH | 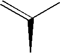 | H | H | —CH₂— | | H | S | S |
| 16 | —OH | 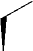 | H | H | — | — | H | S | D |
| 19 | —OH | 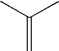 | H | H | —S(C═O)Me | H | H | S | S |
| 22 | —OH | 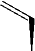 | H | H | H | H | H | S | S |
| 33 | —OH | 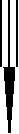 | H | H | H | H | H | S | S |
| 38 | —OH | 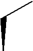 | H | H | H | Me | H | S | S |

S = single bond
D = double bond
— = substituent not present [symbol]
is a single or double bond.

In a second aspect of the invention is provided compounds of the formula I, or a pharmaceutically acceptable ester or ether thereof, for use in therapy.

In a third aspect of the invention is provided a pharmaceutical composition comprising a compound of the formula I, or a pharmaceutically acceptable ester or ether thereof, preferably the said pharmaceutical composition also comprises a pharmaceutically acceptable diluent.

In a fourth aspect of the invention is provided the use, of a compound of formula I, or a pharmaceutically acceptable ester or ether thereof, in the manufacture of a medicament for the treatment of conditions related to the mineralocorticoid receptor.

In a fifth aspect of the invention is provided a method of treatment of conditions associated with the mineralocorticoid receptor, comprising administering to a patient in need thereof, a pharmaceutically effective amount of a compound of formula I, or a pharmaceutically acceptable ester or ether thereof. Preferably the patient is a human patient.

In a sixth aspect of the invention is provided the use of compounds of the formula I as antagonists of the mineralocorticoid receptor. Particularly as antagonists of the mineralocorticoid receptor in vitro. Particularly such compounds may be used conveniently as comparative compounds for the identification of compounds with equal or improved antagonist activity at the mineralocorticoid receptor by comparing the level of antagonist activity of a compound of the formula I with the level of antagonist activity of a test compound.

The level of antagonism of a compound of the formula I or of a test compound may be conveniently determined by comparing the binding of a mineralocorticoid receptor ligand such as aldosterone to the receptor in the presence and in the absence of the compound of the formula I or test compound. Conveniently IC₅₀ values may be calculated (by methods well known in the art and described herein below) for the compound of the formula I and the test compound, and compared.

Medicaments of the invention comprising compounds of the formula I can be administered by oral or parenteral (including intravenous, intramuscular, intraperitoneal, subcutaneous) routes as well as by transdermal, airway (aerosol), rectal, vaginal and topical (including buccal and sublingual) administration.

The medicament may be made up in liquid form, in which case it will typically, in addition to the compound of the formula I, comprise a pharmaceutically acceptable liquid diluent; or it may be made up in solid form and may, in this case, also comprise a solid diluent.

For oral administration, the compounds of the invention will generally be provided in the form of a tablet, hard or soft capsule, a cachet, a troche, a lozenge or capsules, as a powder or granules, or as an aqueous solution or suspension.

Compositions for oral use, such as tablets, may include the active ingredients mixed with pharmaceutically acceptable excipients such as inert diluents, disintegrating agents, binding agents, lubricating agents, sweetening agents, flavouring agents, colouring agents and preservatives. Examples of suitable inert diluents include sodium and calcium carbonate, sodium and calcium phosphate, and lactose, while corn starch and alginic acid are examples of suitable disintegrating agents. Binding agents include, for example starch and gelatine, while the lubricating agent, if present, may for example, be magnesium stearate, stearic acid or talc.

Compositions for oral use may be delivered in a manner which reduces exposure of the composition to selected gut compartments such as the buccal or gastric regions. The composition may be formulated to delay absorption in the gastrointestinal tract for example by coating with an enteric coating material, such as glyceryl mono stearate or glyceryl distearate. Capsules for oral use include hard gelatine capsules in which the active ingredient is mixed with a solid diluent, and soft gelatine capsules wherein the active ingredients is mixed with water or an oil such as peanut oil, liquid paraffin or olive oil

Formulations for rectal administration may for example be presented as a suppository with a suitable base comprising, for example, cocoa butter or a salicylate.

Formulations suitable for vaginal administration may for example be presented as pessaries, tampons, creams, gels, pastes, foams or spray formulations containing in addition to the active ingredient such carriers as are known in the art to be appropriate.

For parenteral use, the compounds of the invention will typically be provided as sterile and pyrogen free preparations. Such preparations will typically comprise a non toxic, parenterally acceptable diluent, to provide, solutions, emulsions, liposome formulations or suspensions. Such preparations may comprise a preservative. Suitable preservatives include ethyl and n-propyl p-hydroxybenzoate for example.

Typically the preparation will be buffered to an appropriate pH and isotonicity. For example suitable diluents include sterile water, Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin.

Compositions for parenteral administration will typically be provided in an ampoule, a multi-dose container or in a single use device for auto injection or injection by a medical practitioner. preparations for multi dosing typically will comprise a preservative.

In general a suitable dose of the compound of the formula I will be in the range of 0.1 mg to 5 mg per kilogram body weight of the recipient per day, preferably in the range of 0.5 mg to 2.5 mg/kg/d. Typically the desired dose is presented once daily or several times a day in sub doses. Conveniently these sub-doses may be administered in unit dosage forms, for example, containing 5 mg to 250 mg, preferably 25 mg to 125 mg, and most preferably 50 mg to 250 mg of active ingredient per unit dosage form.

The present invention will now be described further by reference to the following non-limiting Examples, Schemes and Figures. Further embodiments falling within the scope of the claim will occur to those skilled in the art in the light of these.

### FIGURES

FIGS. 1 to 4 illustrate general methods of synthesis of compounds of the invention.

FIG. 5 illustrates a synthetic route of example 16 (11β)-11-hydroxy-20-methyl-pregna-4,6,17(20)-trien-3-one, example 17 (7α,11β)-7-(acetylthio)-11-hydroxy-20-methyl-pregna-4,17(20)-dien-3-one and other compounds of example 16.

FIG. 6 illustrates a synthetic route to 11β-11-hydroxy-7α-Methyl-pregna-1,4-en-3-one and other compounds of example 18.

FIG. 7 illustrates a synthetic route to example 19 (11β)-11-hydroxypregn-4-en-20-yn-3-one and other compounds of example 19.

FIG. 8 illustrates a synthetic route to example 20 (11β,17Z)-11-hydroxypregna-4,17(20)-dien-3-one and other compounds of example 20.

FIG. 9 illustrates the results of in vivo administration of 11β-11-hydroxy-20-methyl-pregna-4,20-dien-3-one

### GENERAL SYNTHETIC ROUTES

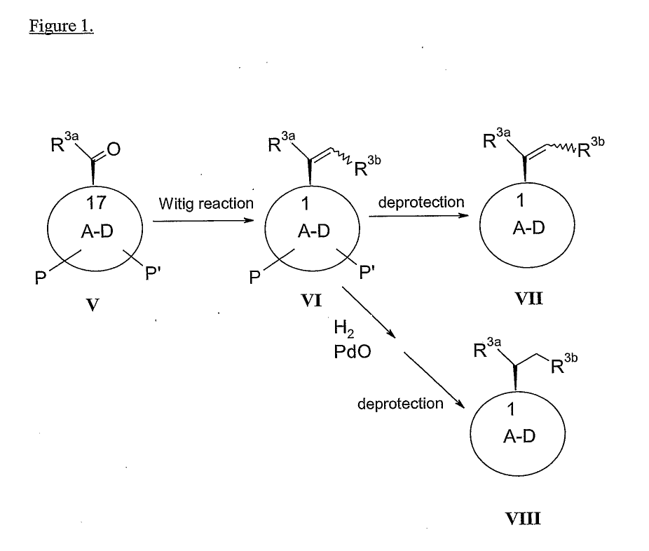

General, synthetic routes are illustrated in FIGS. 1 to 4

Compounds of the present invention can be prepared by general methods well known in the art. For example they may be prepared from compounds of formula V (FIG. 1), wherein A-D represents a steroid skeleton in which reactive functional groups have been protected by protective groups P and P′ (see for example T. W. Grew, protective groups in Organic Synthesis, Wiley, NY, 1981) and in which R^{3a} represents H, halogen, monocyclic aryl or (C₁-C₅)alkyl, optionally substituted with halogen, protected hydroxy, alkoxy or acyloxy.

Condensation of a compound represented by formula V (FIG. 1) with a Wittig (like) compound of the formula R^{3b}═CHPR²⁰ in which R²⁰ is e.g. phenyl, provides compounds of formula VI, which after removal of the protecting group(s) affords the desired compounds of formula VII. R^{3a} and R^{3b} otherwise can be as defined previously. Catalytic hydrogenation of a compound of formula VI, followed by deprotection, affords compounds of formula VIII in which R^{3b} is connected to the remainder of the molecule by a single bond.

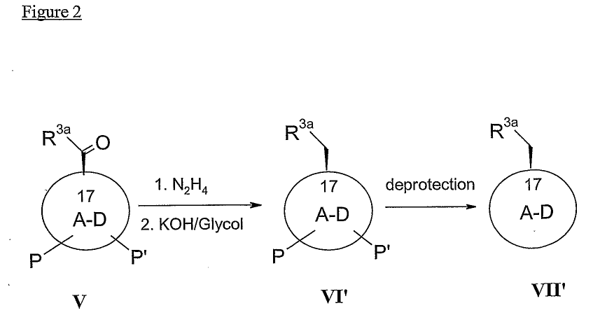

Alternatively (FIG. 2) reduction of compounds of formula V, e.g. via the Wolff Kishner reaction, affords compounds of formula VI′ which upon deprotection afford other compounds of the invention, represented by formula VII′ in which R^{3a} is as defined previously.

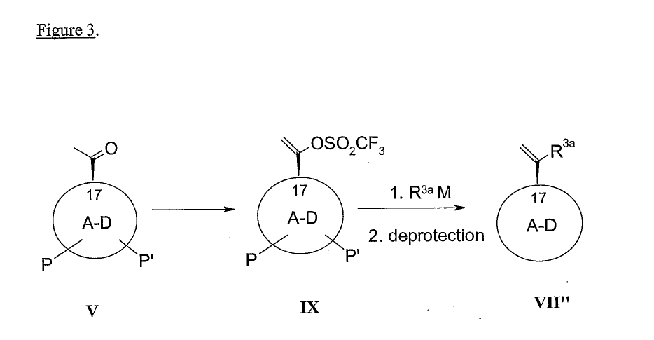

Compounds of formula V with R^{3a}=methyl, (FIG. 3) can also be converted into the enoltriflate derivatives of formula IX by reaction with triflic anhydride and base. Subsequent coupling with an organometallic reagent in the presence of a suitable catalyst, followed by deprotection, affords desired compounds represented by formula VII″. Examples of organometallic reagents are magnesium, manganese, zinc and tin compounds. Among many catalysts representative examples are palladium(0) derivatives and chromium and nickel salts (see i.a. K. Takai et al., Organic Syntheses 72, 180 (1995) F. Orsini et al., Synth. Comm., 17, 1389 (1987), G. Crisp et al., Tetrahedron 50, 3213 (1994)).

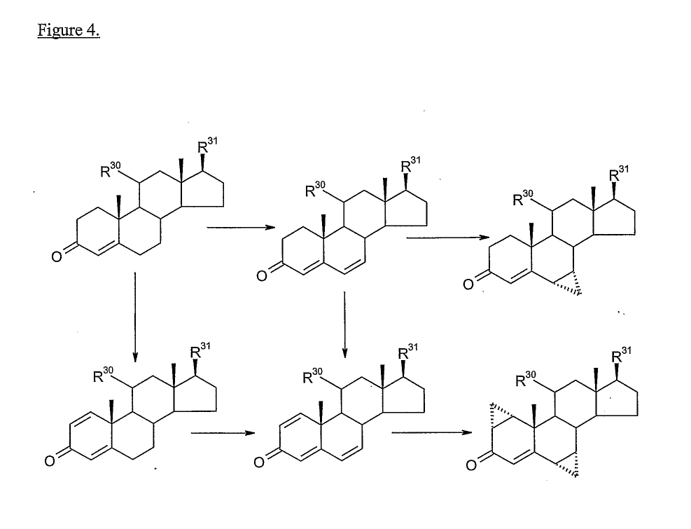

The steroid skeleton, A-D, can be modified at the intermediate stage of the synthesis, or at the end, by known methods. Thus, starting from a steroid with a 4-en-3-one moiety (FIG. 4) additional double bonds can be introduced. As an example, the reaction with chloranil gives a −4,6-dien-3-one moiety, reaction with SeO₂ or DDQ a-1,4-dien-3-one moiety and a combination of the two methods a 1,4,6-trien-3-one moiety. These products can be converted to cyclopropa derivatives by known methods (see e.g. Organic Reactions in Steroid Chemistry, vol. 182, ed. J. Fried and J. A. Edwards, Van Nostrand Reinhold, NY 1972). as shown in the scheme.

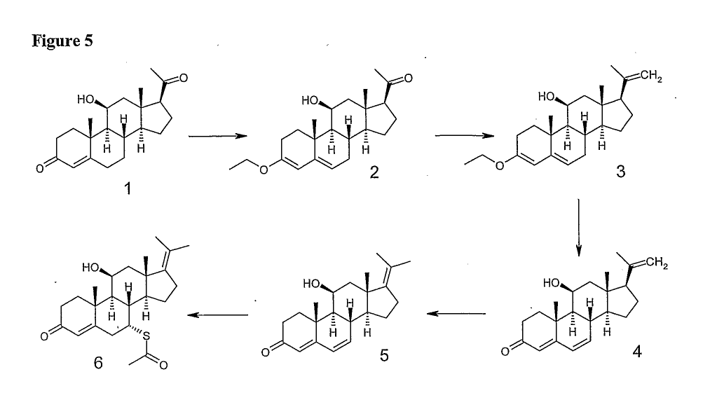

Compounds of example 16 to 20 can be prepared according to the reaction schemes laid out in FIGS. 5 to 8 and in the corresponding experimental sections.

Ester prodrugs can be made by esterification of compounds with free hydroxyl groups by reaction with appropriate acyl chlorides in pyridine.

### SYNTHETIC EXAMPLES

### Example 1

### (11β)-11-hydroxy-20-methylpregna-4,20-dien-3-one

i) To a solution of 11β-hydroxyprogesterone (40.0 g) in dry N,N-dimethylformamide (400 ml) were added 2,2-dimethoxypropane (400 ml), p-toluenesulphonic acid (3.2 g) and methanol (13 ml). The mixture was stirred for 4 h at room temperature and then poured into ice-cold water (4 L), containing 0.5% (v/v) pyridine. The resulting precipitate was collected by filtration and dried in vacuo to give (11β)-11-hydroxy-3-methoxypregna-3,5,-dien-20-one (38.8 g).

¹H NMR (400 MHz, CDCl₃); δ 0.89 (s, 3H. 18-Me), 1.21 (s, 3H, 19-Me), 2.14 (s, 3H, 21-Me), 3.58 (s, 3H, OMe), 4.44 (m, 1H, H-11), 5.09-5.16 (m, 2H, H-4, H-6)

ii) To a suspension of potassium tert-butoxide (9.8 g) in dry toluene (200 ml) was added methyltriphenylphosphonium bromide (35.6 g) under nitrogen. The mixture was allowed to reflux for 1 h. A solution of (11β)-11-hydroxy-3-methoxypregna-3,5,dien-20-one (8.6 g) was added and the resulting mixture was allowed to reflux for 2 h. The reaction mixture was cooled, washed with water (3×30 ml) and concentrated in vacuo. The residue was chromatographed over silica gel with heptane/ethyl acetate 7:3 containing 0.1% (v/v) of triethylamine. This afforded pure (11β)-3-methoxy-20-methylpregna-3,5,20-trien-11-ol (6.7 g).

¹H NMR (400 MHz, CDCl₃): δ 0.85 (s, 3H, 18-Me), 1.21 (s, 3H, 19-Me), 1.78 (br.s, 3H, 20-Me), 3.58 (s, 3H, OMe), 4.39 (m, 1H, H-11), 4.73 and 4.85 (m, 1H each, 2H═CH₂), 5.10 (d, J2, 1H, H-4), 5.15 (t, J4, 1H, H-6).

### Example 2

### (11β,20-E)-11-hydroxy-24-norchola 4,20(22)-dien-3-one

i) A solution of 11β-hydroxyprogesterone (80.0 g) in a mixture of dry abs. ethanol (750 ml) and triethyl orthoformate (100 ml) was cooled to 5° C. p-Toluenesulphonic acid (1.0 g) was added and the resulting mixture was stirred for 1 h. Another portion of p-toluenesulphonic acid (0.5 g) was added and stirring continued for 3 h. The reaction mixture was neutralized with pyridine (50 ml) and then poured into ice-cold water (10 L). The resulting precipitate was collected by filtration and dried in vacuo. The crude product (90 g) was chromatographed over silica gel (500 g) with toluene/ethyl acetate 1:1 to give (11β)-11-hydroxy-3-ethoxypregna-3,5,-dien-20 one (76 g). 1H NMR (400 MHz, CDCl₃): δ 0-89 (s, 3H, 18-Me), 1.21 (s, 3H. 19-Me), 1.31 [t, J7, 3H, Me of ethoxy), 2.14 (s, 3H, 21-Me), 3.78 (q, 2H, OCH₂), 4.44 (m, 1H, H-11), 5.07-5.14 (m, 2H, H-4, H-6).

ii)—To a suspension of potassium tert-butoxide (6.7 g) in dry toluene (150 ml) was added ethyltriphenylphosphonium bromide (26.2 g) under nitrogen. The mixture was allowed to reflux for 1 h. A solution of (11β-11-hydroxy-3-ethoxypregna-3,5,-dien-20-one (6.0 g) in toluene (50 ml) was added and the resulting mixture was allowed to reflux for 2 h. The reaction mixture was cooled to room temperature, aqueous HCl was added (100 ml, 2N) and the resulting mixture was stirred vigorously for 15 min. The reaction mixture was washed with water, aqueous sodium bicarbonate and again with water and concentrated in vacuo. The residue was chromatographed over silica gel with hexane/ethyl acetate 4:1 to give (11β,20-E)-11-hydroxy-24-norchola 4,20 (22)-dien-3-one (2.3 g), mp. 178.8-180° C. (from diethyl ether). [α]_{D}²⁰=+132 (c 1, dioxane), ¹H NMR (400 MHz, CDCl₃): δ 0.82 (s, 3H, 18-Me), 1.45 (s, 3H, 19-Me), 1.61, 1.62 (2×br.s. 3H each, 20- and 22-Me), 4.36 (m, 1H, H-11), 5.28 (m, 1H, 22-H), 5.68 (d, 32, 1H, H-4).

### Example 3

### ((11β)-20E)-21-chloro-11-hydroxy-20-methylpregna-4,2-dien-3-one

i) To a suspension of potassium tert-butoxide (5.1 g) in dry toluene (75 ml) was added chloromethyltriphenylphosphonium bromide (19.0 g) under nitrogen. The mixture was allowed to reflux for 1 h. A solution of (11β)-11-hydroxy-3-ethoxypregna-3,5,-dien-20-one (6.0 g) (example 2.i) in toluene (25 ml) was added and the resulting mixture was allowed to reflux for 2 h. The reaction mixture was cooled to room temperature. aqueous HCl was added (100 ml, 2N) and the resulting mixture was stirred vigorously for 15 min. The reaction mixture was washed with water, aqueous sodium bicarbonate and again with water and concentrated in vacuo.

The residue was chromatographed over silica gel with toluene/ethyl acetate 955 to give pure ((11β)-20-E)-21-chloro-11-hydroxy-20-methylpregna-4,2-dien-3-one (1.7 g), mp. 174.1-176.5° C. (from diethyl ether), [α]D20+136 (c1, dioxane), ¹H NMR (400 MHz, CDCl₃): d 0.87 (s, 3H, 18-Me), 1.45 (s, 3H, 19-Me), 1.81 (d, J 1.5, 3H, 20-Me), 4.38 (m, 1H, H-11), 5.68 (d, 32, 1H, H-4), 5.85 (quint, J 1-3, 1H 22-H).

### Example 4

### (6β,11β)-6-chloro-11-hydroxy-20-methylpregna-4,20-dien-3-one and (6α,11β)-6-chloro-11-hydroxy-20-methylpregna-4,20-dien-3-one

i) To an ice-cold solution of (11β)-11-hydroxy-3-methoxypregna-3,5-dien-20-one (0.5 g) (example 1.i) in acetone (10 ml) was added, with stirring, a solution of sodium acetate (0.17 g) in water (1.25 ml), followed by N-chlorosuccinimid (0.21 g) in small portions and glacial acetic acid (0.15 ml). The mixture was stirred at 0° C. for 2 h, diluted with ice-water and extracted with ethyl acetate (3×50 ml). The combined extracts were washed with water, dried over anhydrous magnesium sulphate and concentrated in vacuo. The residue was chromatographed over silica gel with heptane/ethyl acetate 9:1, containing 0.1% (v/v) of triethylamine. The less polar material consisted of (6β,11β)-6-chloro-11-hydroxy-20-methylpregna-4,20-dien-3-one (0.11 g), mp. 148-150° C. (from diethyl ether). [a]D20=+47 (c 0.2, dioxane), 1H NMR (400 MHz, CDCl3): d 0.91 (s, 3H, 18-Me), 1.73 (s, 3H, 19-Me), 1.77 (br, s, 3H, 20-Me), 4.38 (m, 1H, H-11) 4.72 and 4.87 (m, 1H each, ═CH₂), 4.74 (br.s, 1H, 6-H), 5.85 (br.s, 1H, H-4).

The more polar material consisted of (6α,11β)-6-chloro-11-hydroxy-20-methylpregna-4,20-dien-3-one (0.18 g), mp. 174-177° C. (from diethyl ether). [α]_{D}²⁰+78 (c 0.2, dioxane). 1H NMR (400 MHz, CDCl₃): d 0.86 (s, 3H, 18-Me), 1.47 (s, 3H, 19-Me), 1.76 (br-s, 3H, 20-Me), 4.38 (m, 1 HI H-11), 4.79 (ddd, J2, 5.8, 13, 1H, H-6), 4.73 and 4.87 (m, 1H each, ═CH₂), 8.30 (d, J2, 1H, H-4).

### Example 5

### (6β,11β)-6-fluoro-11-hydroxy-20-methylpregna-4,20-dien-3-one and (6α,11β)-6-fluoro-11-hydroxy-20-methylpregna-4,20-dien-3-one

To a suspension of (11β)-11-hydroxy-3-methoxypregna-3,5,-dien-20-one (0.7 g) (example 1.i) in dry acetonitrile (50 ml) was added 1-fluoropyridinium pyridine heptafluorodiborate (0.75 g) with stirring at, room temperature. The mixture was stirred at room temperature for 5 h, then mixed with water and extracted with ethyl acetate (3×50 ml). The combined extracts were washed with brine, dried over anhydrous magnesium sulphate and concentrated in vacuo. The residue was separated by preparative reverse phase HPLC, using an acetonitrile/water gradient system. This afforded (6β,11β)-6-fluoro-11-hydroxy-20-methylpregna-4,20-dien-3-one (0.04 g). [α]_{D}²⁰=+47 (c 0.2. dioxane). ¹H NMR (400 MHz, CDCl₃): d 0.84 (s, 3H, 18-Me), 1.58 (s, 3H, 19-Me), 1.77 (br-s, 3H; 20-Me), 4.39 (m, 1H, H-11), 4.74 and 4.88 (m, 1H each, ═CH₂), 4.98 (dt, J 49 & 3, 1H 6-H), 5.84 (br.d, J4.4, 1H, H-4).

The more polar material consisted of (6α,11β)-6-fluoro-11-hydroxy-20-methylpregna-4,20-dien-3-one (0.06 g). [α]_{D}²⁰=+104 (c 0.2, dioxane). ¹H NMR (400 MHz, CDCl₃): δ 0.87 (s, 3H, 18-Me), 1.44 (8, 3H, 19-Me), 1.76 (br.s, 3H, 20-Me), 4.38 (m, 1H, H-11), 4.73 and 4.88 (m, 1H each, ═CH₂), 5.20 (dddd, J 2, 6, 12 and 48, 1H, H-6), 6.02 (narrow m, 1H, H-4).

### Example 6

### (11β)-11-hydroxy-20-methypregna-1,4,20-trien-3-one and (11β)-11-hydroxy-20-methypregna-1,4,6,20-tetra-en-3-one

To a solution of (11β)-11-hydroxy-20-methylpregna-4,20-dien-3-one (0.5 g) (example 1) in 1,4-dioxane (50 ml) was added 2,3-dichloro-5,6-dicyano-1,4-benzoquino (0.52 g). The resulting mixture was allowed to reflux overnight, cooled to room temperature and concentrated in vacuo. The residue was dissolved in dichloromethane and repeatedly chromatographed over silica gel with heptane/ethyl acetate 8:2. This afforded pure (11β)-11-hydroxy-20-methylpregna-1,4,20-trien-3-one (0.15 g). [α]_{D}²⁰=+61 (c 0.2, dioxane). ¹HNMR (400 MHz, CDCl₃): δ 0.88 (s, 3H, 18-Me), 1.46 (s, 3H, 19-Me), 1.75 (br.s, 3H, 20-Me), 4.38 (m, 1H, H-11) 4.72 and 4.87 (m, 1H each, ═CH₂), 6.02 (t, J 1.6, 1H, H-4), 6.27 (dd, J 1.7, 10.1H, H-2), 7.28 (d, J 10, 1H, H-1).

From the mother liquor a small amount of (11β)-11-hydroxy-20-methyl-pregna-1,4,6,20-tetra-en-3-one (0.05 g) was obtained via further chromatographic separation.

### Example 7

### (11β)-11-hydroxy-20-methylpregna-4,6,20-trien-3-one

A solution of (11β)-11-hydroxy-3-methoxypregna-3,5,-dien-20-one (1.0 g) (example 1.i) in dichloromethane (2.5 ml) was added to a suspension of chloranil in a mixture of methanol (6.2 ml), water (0.33 ml), dichloromethane (2.5 ml), acetic acid (0.62 ml) and pyridine (0.074 ml). The resulting mixture was stirred for 1 h at room temperature. A solution of sodium hydroxide (0.26 g) and sodium dithionite (0.26 g) in water (10 ml) was added and the resulting mixture was stirred vigorously for 15 min. The reaction mixture was extracted with dichloromethane (3×50 ml) and the combined extracts were washed with N aqueous sodium hydroxide and with water until neutral. On concentration the product crystallized from the solution giving (11β)-11-hydroxy-20-methylpregna-4,6,20-trien-3-one (0.7 g), mp. 153-155° C. ¹H NMR (400 MHz, CDCl₃); δ 0.92 (s, 3H, 18-Me), 1.37 (s, 3H, 19-Me), 1.77 (br.s, 3H, 20-Me), 4.37 (m, 1H, H-11), 4.74 and 4.88 (m 1H each, ═CH₂), 5.63 (s, 1H, H-4), 6.10 (dd, J 2.3, 9.6, 1H, H-7), 6.22 (dd, J 1.9, 9.6, 1H, H-6).

### Example 8

### (6α7α11β)-11-hydroxy-20-methylcyclopropa[6,7]pregna-4,20-dien-3-one

To a suspension of sodium hydride (0.184 g, 60% in mineral oil) in dry dimethylsulphoxide (4.2 ml) was added a solution of trimethylsulphoxonium iodide (1.0 g) in dimethylsulphoxide (3 ml) under nitrogen with stirring and slight cooling. The resulting mixture was stirred for 1 h at room temperature. A solution of (11β)-11-hydroxy-20-methylpregna-4,6,20-trien-3-one (0.50 g) (example 7) in dimethyl-sulphoxide (3 ml) was added dropwise with stirring. After continued stirring for 3 days at room temperature the reaction mixture was diluted with water, neutralized with 2N aqueous hydrochloric acid and extracted with ethyl acetate (3×30 ml). The organic extracts were washed with water and brine and dried over anhydrous magnesium sulphate, and concentrated in vacuo. The residue was chromatographed over silica gel with toluene/ethyl acetate 8:2. This afforded pure (6α,7α,11β)-11-hydroxy-20-methylcyclopropa[6,7]pregna-4,20-dien-3-one (0.10 g). [α]_{D}²⁰=−50.5 (c 1.8, dioxane). ¹H NMR (400 MHz, CDCl₃): δ 0.86 (s, 3H, 18-Me), 1.33 (s, 3H, 19-Me), 1-77 (br.s, 3H, 20-Me), 4.31 (m, 1H, H-11), 4.74 and 4.88 (m, 1H each, ═CH₂), 5.94 (s, 1H, H-4).

### Example 9

### (11β)-11-hydroxy-20-hydroxymethypregna-4,20-Bien-3-one

i) To a solution of corticosterone (10.4 g) in dry N,N-dimethylformamide (80 ml) were added 2,2-dimethoxypropane (80 ml), p-toluenesulphonic acid (0.80 g) and methanol (33 ml). The mixture was stirred for 6.5 h at room temperature and then poured in ice-cold water (1 L], containing 0.5% (v/v) of pyridine. The resulting precipitate was collected by filtration and dried in vacuo to give sticky crystalline material, This was purified by chromatography over silica gel with toluene/ethyl acetate 8:2 to give pure (11β)-11,21-dihydroxy-3-methoxypregna-3,5-dien-20-one. (4.7 g). ¹H NMR (400 MHz, CDCl₃): δ 0.92 (s, 3H, 18-Me), 1.21 (s, 3H, 19-Me), 3.58 (s, 3H, OMe), 4.20 (m, 2H, CH₂O), 4.44 (m, 1H, H-11) 5.09-5.16 (m, 2H, H-4, H-6).

ii)—To a suspension of potassium tert-butoxide (5.5 g) in dry toluene (100 ml) was added methyltriphenylphosphonium bromide (20.0 g) under nitrogen. The mixture was allowed to reflux for 1 h. A solution of (11β)-11,21-dihydroxy-3-methoxypregna-3,5,-dien-20-one (3.3 g) in toluene (15 ml) was added and the resulting mixture was allowed to reflux for 2 h. The reaction mixture was cooled, washed with water (3×30 ml) and concentrated in vacuo. The residue was purified by chromatography over silica gel with toluene/ethyl acetate 8:2 containing 0.1% (v/v) of triethylamine to give pure (11β)-3-methoxy-20-hydroxymethylpregna-3,5,20-triene-11-ol (1.6 g). ¹H NMR (400 MHz, CDCl₃): δ 0.86 (s, 3H, 18-Me), 1.21 (s, 3H, 19-Me), 3.58 (s, 3H, OMe), 4.09 (m, 2H, CH₂O), 4.39 (m, 1H H11), 4.98 and 5.21 (m, 1H each, ═CH₂), 5.09-5.16 (m, 2H, H-4, H-6).

iii)—(11β)-3-methoxy-20-hydroxymethylpregna-3,5,20-triene-11-ol (1.6 g) was dissolved in acetone (40 ml) and cooled to 0° C. 2N aqueous HCl (1.0 ml) was added and the resulting solution was stirred for 30 min. The reaction mixture was neutralized with pyridine and poured into ice cold water (300 ml). The precipitate was collected by filtration, dried and triturated with diethyl ether to give pure (11β)-11-hydroxy-20-hydroxymethypregna-4,20-dien-3-one. (1.14 g). [α]_{D}²⁰=+1.48 [c 0.4, dioxane). ¹HNMR (400 MHz, CDCl₃): δ 0.88 (s, 3H, 18-Me), 1.45 (s, 3H, 19-Me), 4.07 (m, 2H, CH₂O), 4.37 (m, 1H, H-11), 4.98 and 5.22 (m, 1H each, ═CH₂), 5.68 (d, J2, 1H, H-4).

### Example 10

### (11β)-20-[(acetyloxy)methyl]-11-hydroxypregna-4,20-dien-3-one

Acetic anhydride (0.70 ml) was added dropwise with stirring to a solution of (11β)-11-hydroxy-20-hydroxymethylpregna-4,20-dien-3-one (0.33 g) (example 9) in dry pyridine (1.6 ml) at room temperature. The resulting solution was stirred for 16 h at room temperature, diluted with water (20 ml) and extracted with ethyl acetate (3×10 ml). The combined extracts were washed with water, followed by aqueous sodium bicarbonate and dried over anhydrous magnesium sulphate. Evaporation of the solvent and crystallisation from diisopropyl ether gave pure (11β)-20-[(acetyloxy)methyl]-11-hydroxypregna-4,20-dien-3-one (0.10 g). [α]_{D}²⁰=+1.36 (c 0.1, dioxane). ¹HNMR (400 MHz, CDCl₃): δ 0.89 (5, 3H, 18-Me), 1.46 (s, 3H, 19-Me), 2.70 (s, 3H, acetyl), 4.37 (m, 1H, H-I I) 4.51 (m, 2H, CHZO), 5.02 and 5.19 (m, 1H each, ═CHS, 5.68 (d, J 2, IH, H-4).

### Example 11

### (11β)-20-[(chloro)methyl]-hydroxypregna-4,20-dien-3-one

To a solution of (11β)-11-hydroxy-20-hydroxymethylpregna-4,20-dien-3-one (0.33 g) (example 9) in dry pyridine (4 ml) was added p-toluenesulphonyl chloride (1.06 g) at room temperature. The resulting solution was stirred for 16 h at room temperature, diluted with water (40 ml), acidified with 2N aqueous hydrochloric acid (10.0 ml) and extracted with ethyl acetate (3×30 ml). The combined extracts were washed with brine, dried over anhydrous magnesium sulphate and concentrated in vacuo. The residue was purified by chromatography over silica gel with toluene/ethyl acetate 8:2 containing 0.1% (v/v) of triethylamine to give pure (11β)-20-[(chloro)methyl]-hydroxypregna-4,20-dien-3-one (0.055 g). [α]_{D}²⁰=−+1.09 (c 0.1, dioxane). ¹H NMR (400 MHz, CDCl₃): δ 0.88 (s, 3H, 18-Me), 1.45 (s, 3H, 19-Me), 4.06 {s, 2H, CH₂Cl), 4.38 (m, 1H, H-11), 5.07 and 5.32 (m, 1H each, ═CH₂), 5.69 (d, J2, 1H H-4).

### Example 12

### (6β,11β)-6-chloro-11-hydroxypregna-1,4-dien-3-one

By a similar procedure to that described in examples 4 (11β)-20-[(chloro)methyl]-hydroxypregna-4,20-dien-3-one-3-methoxypregna-3,5-dien-11-ol was converted into (6α,11β)-6-chloro-11-hydroxypregna-1,4-dien-3-one and (6β,11β)-6-chloro-11-hydroxypregna-1,4-dien-3-one.

### Example 13

### (6β,11β)-6-fluoro-11-hydroxypregna-1,4-dien-3-one

By a similar procedure to that described in example 5 (11β)-3-methoxypregna-3,5-dien-11-ol was converted into (6α,11β)-6-fluoro-11-hydroxypregna-1,4-dien-3-one and (6β,11β)-6-fluoro-11-hydroxypregna-1,4-dien-3-one.

### Example 14

### (11β)-11-hydroxypregna-1,4-dien-3-one and (11β)-11-hydroxypregna-1,4,6-trien-3-one

By a similar procedure to that described in example 6, (11β)-11-hydroxypregn-4-en-3-one was converted into (11β)-11-hydroxypregna-1,4-dien-3-one and (11β)-11-hydroxypregna-1,4,6-trien-3-one

### Example 15

### (11β)-11-hydroxypregn-4,6-dien-3-one

By a similar procedure as described in example 7 (11β)-11-hydroxypregn-4-en-3-one was converted into (11β)-11-hydroxypregn-4,6-dien-3-one.

### Example 16

### (11β)-11-hydroxy-20-methyl-pregna-4,6,17(20)-trien-3-one

### Compound 2

i) To a solution of 11β-hydroxypregn-4-en-3-one (1, FIG. 5) (5 g) in a mixture of dry ethanol (203 mL) and triethyl orthoformate (4.41 mL) was added p-toluenesulphonic acid (92 mg) under nitrogen and stirred for 2 h. Another portion of p-toluenesulphonic acid (92 mg) was added and stirring continued overnight. The reaction mixture was neutralized with aqueous sodium bicarbonate and extracted with ethyl acetate and washed with water and brine. The organic layer was dried over anhydrous sodium sulphate and concentrated in vacuo to give (11β)-11-hydroxy-3-ethoxypregnane-3,5-dien-20-one (2) (5.4 g).

### Compound 3

ii) To a suspension of potassium tert-butoxide (3.48 g) in dry toluene (150 mL) methyltriphenylphosphonium bromide (15.3 g) was added under nitrogen. The reaction mixture was allowed to reflux for 1 h. A solution of (11β)-11-hydroxy-3-ethoxypregnane-3,5,-dien-20-one (2) (5.4 g) in toluene (60 mL) was added and the resulting mixture was allowed to reflux for an additional 2 h. The reaction mixture was cooled to 0° C., water was added and the reaction mixture was stirred vigorously for 15 min, then extracted with ethyl acetate, washed with water, aqueous sodium bicarbonate and brine and concentrated in vacuo. The residue was dissolved in dichloromethane and added drop wise to cooled (0° C.) heptane. After concentrating in vacuo crystallization of triphenylphosphineoxide started. The filtrate was chromatographed over silica gel with heptane/ethyl acetate 9:1 containing 0.1% (v/v) of triethylamine to give pure 3 (3.26 g).

### Compound 4

iii) To a solution of compound 3 (3.26 g) in tetrahydrofuran (7.1 mL) and pyridine (0.1 mL) was added a suspension of chloranil (2.29 g) in a mixture of ethanol (16.1 mL) and water (1.94 mL). The resulting mixture was stirred for 2 h at room temperature. Aqueous sodium bicarbonate was added and the resulting mixture was stirred vigorously for 15 min. The reaction mixture was extracted with dichloromethane and washed with aqueous sodium bicarbonate (3×), brine and concentrated in vacuo. The crude product was chromatographed over silica gel with heptane/ethyl acetate 8:2 containing 0.1% (v/v) of triethylamine to give compound 4 (1.99 g).

### (11β)-11-hydroxy-20-methyl-pregna-4,6,17(20)-trien-3-one

iv) A solution of compound 4 (FIG. 5) (1.99 g) in acetone (100 mL) and hydrochloric acid (10.2 mL, 6 M) was allowed to reflux for 6 h followed by stirring at room temperature overnight. The reaction mixture was neutralised by pouring in ice-cold aqueous sodium bicarbonate after cooling to room temperature. The resulting mixture was extracted with ethyl acetate, water and brine and dried over anhyd. sodium sulphate and concentrated in vacuo. Chromatography using silica gel (heptane/ethyl acetate, 8:2) gave the desired (11β)-11-hydroxy-20-methyl-pregna-4,6,17(20)-trien-3-one (5) (1.04 g). ¹H NMR (CDCl₃): δ 1.07-1.55 (m, 5H), 1.16 (s, 3H), 1.38 (s, 3H), 1.56 (s, 3H), 1.69 (s, 3H), 1.70-1.95 (m, 3H), 2.18-2.46 (m, 4H), 2.58-2.72 (m, 2H) 4.37-4.41 (m, 1H) 5.63 (s, 1H), 6.10-6.14 (dd, 1H), 6.26-6.30 (dd, 1H).

### Example 17

### (7α,11β)-7-(acetylthio)-11-hydroxy-20-methyl-pregna-4,17(20)-dien-3-one

v) To a solution of compound 5 (FIG. 5), (1.04 g) in dry tetrahydrofuran (208 mL) was added thiolacetic acid (728 μL) followed by trimethylsilyl trifluoromethanesulphonate (208 μL) under nitrogen. The reaction was stirred for 3 days at room temperature. The reaction mixture was neutralised with aqueous sodium bicarbonate and extracted with ethyl acetate, washed with water and brine and concentrated in vacuo. The residue was chromatographed (silica gel, heptane/ethyl acetate, 8:2) followed by crystallization from dichloromethane/heptane, which gave the desired pure (7α,11β)-7-(acetylthio)-11-hydroxy-20-methyl-pregna-4,17 (20)-dien-3-one (6) (250 mg). ¹H NMR (CDCl₃): δ 1.00-1.09 (m, 2H), 1.12 (s, 3H), 1.22-1.41 (m, 2H), 1.50 (s, 3H), 1.57 (s, 3H), 1.70 (st, 3H), 1.71-1.77 (m, 1H), 1.81-1.92 (m, 1H), 2.21-2.55 (m, 8H), 2.33 (s, 3H), 2.90-2.97 (m, 1H), 4.09-4.13 (m, 1H), 4.36-4.40 (m, 1H), 5.64 (sd, 1H).

### Example 18

### 11β-11-hydroxy-7α-Methyl-pregna-1,4-en-3-one

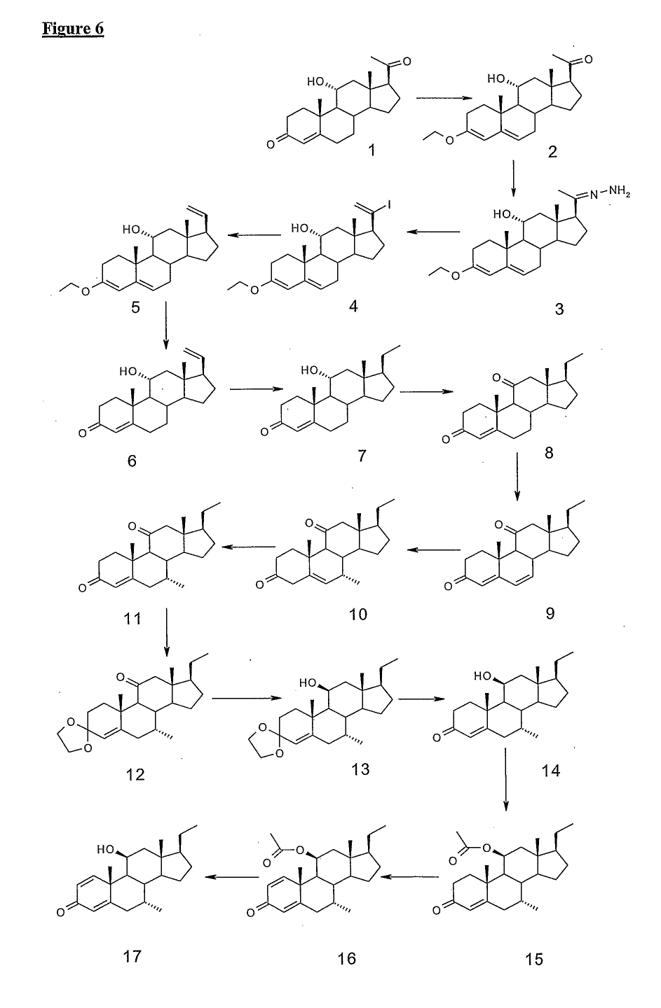

11β-11-hydroxy-7α-Methyl-pregna-1,4-en-3-one was synthesised according to the reaction scheme laid out in FIG. 6.

i) To a solution of (11α)-11-hydroxypregn-4-en-3-one (1, FIG. 6) (10.58 g) in a mixture of dry abs. ethanol (423 ml) and triethyl orthoformate (9.31 ml) was added p-toluenesulphonic acid (201 mg) under nitrogen and stirred overnight at room temperature. The reaction was neutralised with aqueous sodium bicarbonate and extracted with ethyl acetate and washed with water and brine. The organic layer was dried over anhydrous sodium sulphate and concentrated in vacuo to give crude (11α)-11-hydroxy-3-ethoxypregnane-3,5,-dien-20-one (2) (13.4 g).

### Compound 3.

ii) To a solution of compound 2 (3.84 g) in abs. ethanol (30 ml) and triethylamine (16 ml) was added hydrazine hydrate (5.2 ml). The reaction mixture was refluxed for 1.5 h and poured into ice-water and extracted with ethyl acetate, water and brine. The organic layer was dried over anhydrous sodium sulphate and concentrated in vacuo. The residue was purified over silica gel with toluene/ethyl acetate 9:1 containing 0.1% (v/v) of triethylamine to give compound 13 (2.75 g).

### Compound 4.

iii) Compound 3 (2.72 g) was dissolved in THF (181 ml) and triethylamine (77.9 ml) at mom temperature under nitrogen. A solution of iodine (4.87 g) in tetrahydrofuran (41 ml) was added drop wise and allowed to stir for 1 h at room temperature. The reaction mixture was neutralised by the addition of aqueous sodium thiosulfate and extracted with ethyl acetate. The organic layer was washed with water and brine, dried over anhydrous sodium sulphate and concentrated in vacuo. The residue was purified over silica gel with toluene/ethyl acetate 8:2 to give 14 (1.39).

### Compound 5.

iv) To a solution of compound 4 (1.3 g) in tetrahydrofuran at −78° C. was added drop wise n-butyl lithium (4.37 ml, 1.6M) under nitrogen. The reaction mixture was allowed to warm up to room temperature in 1 h and stirred at this temperature for 30 min. After cooling to 0° C. aqueous ammonium chloride was added and stirred for 15 min. The resulting mixture was extracted with ethyl acetate and washed with water and brine. The organic layer was dried over anhydrous sodium sulphate and concentrated in vacuo to give compound 5 (1.3 g).

### Compound 6.

v) Compound 5 (1.3 g) was dissolved in acetone (88 ml) and hydrochloric acid (2.1 ml, 2.0M) was added under nitrogen and allowed to stir for 1 h at room temperature. The reaction mixture was neutralised with aqueous sodium bicarbonate and extracted with ethyl acetate and washed with water and brine. The organic layer was dried over anhydrous sodium sulphate and concentrated in vacuo. The residue was purified over silica gel with toluene/ethyl acetate 7:3 to give compound 6 (500 mg).

### Compound 7.

vi) To a solution of compound 16 (25 mg) in absolute EtOH was added Wilkinson's catalyst ((PPh₃)₃RhCl, 10 mg). The mixture was hydrogenated at atmospheric pressure and room temperature for 6 h. The mixture was filtered over decalite then concentrated in vacuo. The residue was purified over silica gel with heptane/ethyl acetate 6:4 to give compound 7 (22 mg).

### Compound 8.

vii) Jones reagent (6 ml) was added to cooled solution of 17 (6 g) in acetone (12 ml) keeping the reaction temperature below 15° C. After stirring for 15 min. the reaction was worked up in the normal manner. (Excess reagent was destroyed with isopropanol and filtered to remove the chromium salts.) The crude mixture was passed through a short silica column to yield 2.7 g of crystalline compound 8.

### Compound 9.

viii) To compound 18 (10 g) in anhydrous dioxan (100 mL) was added DDQ (8.67 g). HCl gas bubbled through the solution until an excess was present then stirred for 20 mins. DDHQ was removed from the reaction by filtration and the dioxan solution poured into water (100 ml) to afford a solid which was filtered and washed until almost neutral. The solid was dissolved in ethyl acetate and neutralised with carbonate (NB.: use of dichloromethane to dissolve the solid results in the formation of an emulsion). The organic layer was dried over sodium sulphate and the solvent removed. The residue was dissolved in dichloromethane and passed through a short alumina column to remove the remaining DDQ. Chromatography on silica, eluent was toluene/ethyl acetate 1:1, to give 7 g of the Δ^{4,6}-derivative, 9.

### Compound 11.

ix) Methyl magnesium bromide (75 ml. 0.6M) was cooled in an ice bath and cupric acetate (1.13 g) was added keeping the temperature below 15° C. The temperature was lowered to 0° C. while the Δ^{4,6}-derivative in anhydrous THF (40 ml) was added dropwise. Once the addition was complete the reaction was stirred for 10 min before being poured into water (1000 ml) containing ammonium chloride. The product was extracted into EtOAc, dried, then evaporated to dryness to afford compound 10.

Crude 10 was dissolved in methanol (20 ml) and aq. KOH (1 ml, 10N) added. After 45 minutes the reaction was complete by TLC. The caustic was neutralised with acetic acid. Removal of the MeOH gave a gum which was dissolved in EtOAc and washed with water, 6.8 g on removal of the solvent. The product was purified by chromatography on silica (toluene to toluene/ethyl acetate 2:1) to afford 4.39 of compound 11.

### Compound 12.

x) A solution of compound 21 (5 g), ethylene glycol (10 mL), triethyl orthoformate (5 ml) and PTSA (0.25 g) in anhydrous dichloromethane (40 ml) were heated under reflux. After 1 h the reaction was still incomplete so a further 2.5 ml triethyl orthoformate was added, 15 min later there was no starting material remaining. After cooling the solution was neutralised with pyridine. The dichloromethane was removed and a mixture of EtOAc and water added. The organic layer was dried then evaporated to dryness to yield 5.1 g of compound 12.

### Compound 13.

xi) The ketone 12 (4.1 g) was dissolved in MeOH (10 ml) and THF (10 ml), NaBH₄ was added portion-wise, the solution increased in temperature until it refluxed. The reaction was complete after 30 min. Excess borohydride was destroyed with AcOH before the solvent was removed. The residue was partitioned between water and ethyl acetate and the organic layer dried then evaporated to dryness to give a red gum (3.1 g) of 13.

### Compound 14.

xii) A solution of ketal 23 (5 g) in MeOH (20 ml) and aq. HCl (1 ml, 2N) was allowed to stand overnight. Sodium acetate was added and the MeOH removed, the residue was dissolved in EtOAc and washed with water. The ketone was purified by chromatography on silica (toluene-->toluene/EtOAc 2:1) to give 2.1 g of compound 14.

### Compound 15.

xiii) Compound 14 (2.4 g), DMAP (120 mg) and acetic anhydride (1.2 ml) in pyridine (4.8 ml) was allowed to stand overnight. The reaction mixture was poured onto ice. When the ice had melted the mixture was filtered through a cotton wool plug and the gum collected was dissolved in EtOAc before washings with water, 2N HCl, water, aq. Na₂CO₃ then water. Removal of the solvent gave compound 15 as a yellow gum (2.6 g).

### Compound 16.

xiv) Compound 15 (2.6 g), DDQ (1.74 g) and acetic acid (3 ml) in toluene (30 ml) were heated under reflux for 2.5 h. DDHQ was filtered of and the filtrate washed with water, then aq. carbonate. The organic layer was separated off and dried, then evaporated to dryness. The gum was dissolved in dichloromethane and passed through a short alumina column before being purified on silica (Toluene/EtOAc 1:1), to afford 1.5 g of compound 16.

### 11β-11-hydroxy-7α-Methyl-pregna-1,4-en-3-one (17)

The 11β-acetate, 16, (1.5 g) in aqueous KOH (6 ml 10N) and methanol (30 ml) was heated under reflux for 1.5 h. After cooling the reaction was neutralised with AcOH then poured into water. The resulting solid was filtered and dissolved in dichloromethane, dried over sodium sulfate and evaporated to dryness, weight of crude product was 1.1 g. The product was crystallised from MeOH to give 0.8 g of 11β-11-hydroxy-7α-Methyl-pregna-1,4-en-3-one (17).

¹H NMR (CDCl₃): δ 0.74-0.78 (d, 3H), 0.85-0.90 (t, 3H), 0.87 (s, 3H), 0.97-1.45 (m, 10H), 1.48 (s, 3H), 1.61-1.73 (m, 1H), 1.81-1.90 (m, 2H), 2.05-2.23 (m, 3H), 2.79-2.85 (m, 1H), 4.38-4.42 (m, 1H), 5.99 (st, 1H) 6.24-6.29 (dd, 1H), 7.28-7.33 (d, 1H).

### Example 19

### (11β)-11-hydroxypregn-4-en-20-yn-3-one

### Compound 3

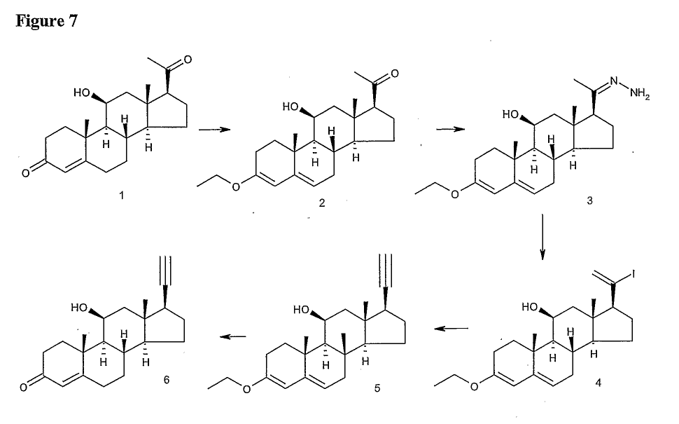

i) To a stirred solution of compound 2 (FIG. 7; prepared from compound 1 as described in example 16) (820 mg) in EtOH (6.2 mL) and Et₃N (3.4 mL), hydrazine.hydrate (1.11 mL) was added and heated to reflux temperature. After 4 hours the reaction mixture was poured into H₂O and the product was extracted into EtOAc. The organics were washed with H₂O and brine, dried (anhydr. Na₂SO₄) and concentrated in vacuo to give compound 3 (769 mg, 90%). The product was used without further purification.

### Compound 4

ii) To a stirred solution of compound 3 (569 mg, 1.53 mmol) in THF (38 mL) and Et₃N (16.3 mL), a solution of iodine (1.02 g) in THF (8.6 mL) was added dropwise. The resulting mixture was stirred for 1.5 hours at room temperature. Then the reaction mixture was poured into a saturated aqueous Na₂SO₃ solution and the product was extracted into EtOAc. The organics were washed with brine, dried and concentrated under reduced pressure. The crude product was purified on silica to give compound 4 (554 mg, 77%).

### Compound 5

iii) To a stirred solution of compound 4 (174 mg, 0.37 mmol) in tert-BuOH (7 mL), KOtBu (620 mg) was added. The reaction was heated to reflux temperature and stirred overnight. Then the reaction was poured into a saturated aqueous NH₄Cl solution. The product was extracted into EtOAc. The organics were washed with a saturated aqueous NH₄Cl solution and brine, dried and concentrated under reduced pressure to give compound 5 (93 mg, 73% crude) which was used without further purification.

### Compound 6

iv) To a stirred solution of 5 (93 mg) in aceton (8.6 mL) was added 2N HCl (151 μl). After stirring for 1 hour at room temperature the reaction mixture was poured into a saturated aqueous NaHCO₃ solution and the product was extracted into EtOAc. The product was purified on silica (Hept:EtOAc, 7:3) followed by preparative HPLC separation (acetonitrile/water 60-100% in 30 min.) to give (11β)-11-hydroxypregn-4-en-20-yn-3-one (6) (FIG. 7) (7.8 mg, 9%). ¹H NMR (CDCl₃): δ 0.90-1.09 (m, 3H), 1.10 (s, 3H), 1.33-1.41 (m, 2H); 1.46 (s, 3H), 1.71-2.54 (m, 14H), 4.38-4.42 (m, 1H), 5.68 (sd, 1H).

### Example 20

### (11β,17Z)-11-hydroxypregna-4,17(20)-dien-3-one

### Compound 3

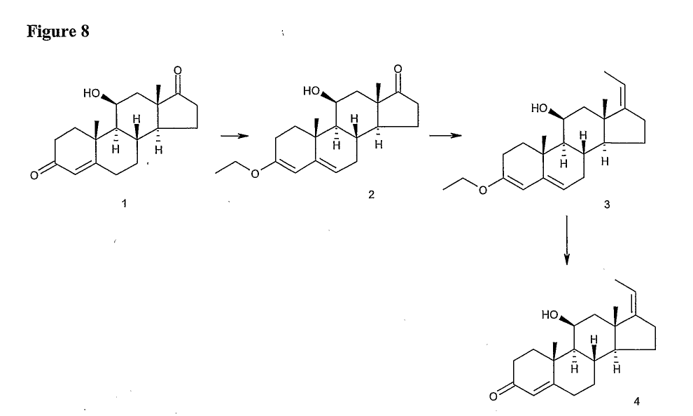

i) To a suspension of potassium tert-butoxide (100 mg) in dry toluene (3 mL) ethyltriphenylphosphonium bromide (370 mg) under nitrogen was added (FIG. 8). Then the reaction mixture was refluxed for 1 h. A solution of compound 2 (50 mg, prepared from (11β)-11-hydroxyandrost-4-ene-3,17-dione (1) according to the general procedure as described in e.g. example 16) in toluene (1 mL) was added and the resulting reaction mixture was refluxed for an additional hour. The reaction mixture was cooled to room temperature and poured into water. The product was extracted into ethyl acetate, washed with brine and concentrated in vacuo. The crude product was purified on silica (Heptane:EtOAc 95:5 containing 0.1% (v/v) Et₃N) to give compound 3 (18 mg, 35%).

### Compound 4

ii) To a stirred solution of compound 3 (18 mg, 0.053 mmol) in aceton (2 mL) HCl (2N, 53 μl) was added. After stirring for 1 hour at room temperature the reaction was poured into saturated NaHCO₃ solution and the product was extracted into EtOAc. The product was purified on silica (Hept:EtOAc, 7:3) followed by preparative HPLC separation (acetonitrile/water 30-100%, in 45 min.) to give (11β,17Z)-11-hydroxypregna-4,17(20)-dien-3-one (4) (7 mg, 42%). ¹H NMR (CDCl₃): δ 0.98-1.18 (m, 3H), 1.16 (s, 3H), 1.28-1.40 (m, 1H), 1.45 (s, 3H), 1.62-2.54 (m, 16H), 4.38-4.42 (m, 1H) 5.06-5.14 (m, 1H), 5.68 (sd, 1H).

### IN VITRO EXAMPLE

### Example 21

### In Vitro Binding to Mineralocorticoid Receptor (MR)

In vitro binding to the mineralocorticoid receptor (MR) was essentially carried out as reported in the literature (C. E. Gomez-Sanchez and E. P. Gomez-Sanchez, Endocrinology 113 (1983) 1004-1009, Z. S. Krozowski and J. W. Funder, Proc. Natl. Acad. Sci. USA 80 (1983) 6056-; J. H. M. H. Reul and E. R. de Kloet, Endocrinology 117 (1985) 2505-2511; K. E. Sheppard and J. W. Funder, J. Steroid Biochem. 28 (1987) 737-742).

Male Wistar rats Were adrenalectomised and perfused with cold saline after 3 days of survival. Kidneys and hippocampi, respectively, were rapidly dissected and homogenized. A cylosolic fraction was prepared by centrifugation. For MR binding studies aliquots of cytosol were incubated with tritiated aldosterone (1 nM).

Unlabeled RU 28362 (10 nM), a specific GR binder, was added for saturating glucocorticoid receptors. Compounds to be tested, dissolved in DMF and diluted with water to obtain the required concentration, were added in an increasing concentration range (1.0-1000 nM) and allowed to incubate for 3 h. Incubations were terminated by adding dextran-coated charcoal suspension, followed by centrifugation. The supernatant was assayed with a scintillation counter for unbound labelled ligand. Non-specific binding was assayed by adding excess of unlabelled aldosterone. Concentrations of test compound displacing 50% of aldosterone (IC₅₀s) are presented in table 2

TABLE 2

| | Mineralocorticoid receptor |
|---|---|
| Compound | Antagonist activity IC₅₀ (nM) |
| Aldosterone | 4 |
| Corticosterone | 5 |
| Spironolactone | 6 |
| (11β)-11-hydroxy-20-methylpregna- | 0.44 |
| 4,20-dien-3-one (synthetic example 1) | |
| (11β)-11-hydroxypregna-1,4-dien-3- | 0.12 |
| one (synthetic example 14) | |

(data given are for hippocampal cytosol):

IN VIVO EXAMPLE

### Example 22

### In Vivo Anti-Mineralocorticoid Activity of (11β-11-hydroxy-20-methyl-pregna-4,20-dien-3-one

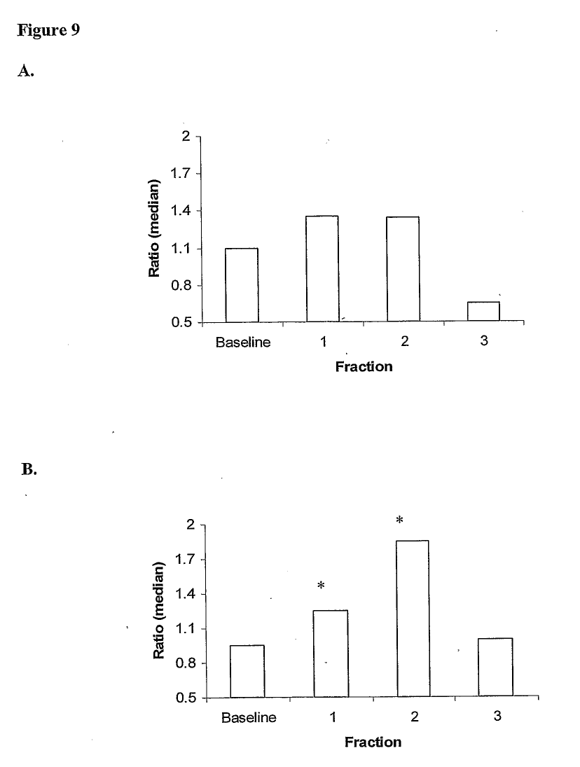

Male rats (weight approx. 100 g) were adrenalectomized on day 4 of the experiment. On day 5 the treatment groups were given a single oral dose of 32 mg/kg of (11β)-11-hydroxy-20-methylpregna-4,20-dien-3-one (compound made in example 1) at 08.00 h, followed by a single dose of aldosterone (2 pg/kg/sc) at 09.00 h. The control group was only given a single dose of aldosterone (2 pg/kg/sc) at 09.00 h. Urine was collected from 2 h periods and the electrolyte excretion (Na⁺/K⁺ ratio) were determined. Results are shown in FIG. 9. Therein FIG. 9A shows results with the control group and FIG. 9B shows results with the experimental group. On the vertical axis is Na⁴/K⁺ ratio in urine and each bar represents results from urine samples collected after one of 4 time points: baseline, which is the urine produced during the night, and fractions 1, 2 and 3 from 0-2, 2-4 and 4-6 hours after aldosterone injection.

1. A compound having a steroid skeleton and substitution characteristics in the A and B rings of the steroid skeleton effective for mineralocorticoid receptor antagonism, and rings C and D of the steroid skeleton having substituents thereon according to formula I 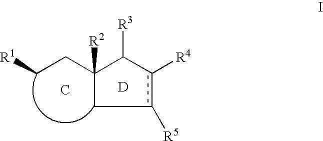
wherein:
R¹ is —OH or ═O;
R² is (C₁₋₃)alkyl or (C₂₋₃)alkenyl;
R³ is selected from: 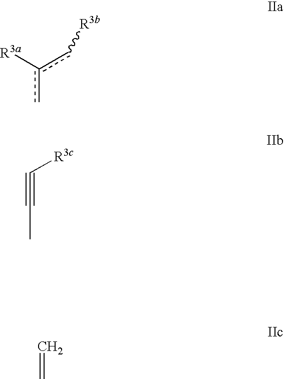
Wherein the lowermost carbon is carbon 17 of the D ring.
R^{3a} is H, halogen, monocyclic aryl or is (C₁₋₅)alkyl optionally substituted with hydroxy, halogen, (C₁₋₆)alkoxy or (C₁₋₆)acyloxy;
R^{3b} is H, (C₁₋₃)alkyl or halogen; and
R^{3c} is H, (C₁₋₆)alkyl, (C₂₋₆)alkenyl or (C₂₋₆)alkynyl;
R⁴ is H or (C₁₋₆)alkyl;
R⁵ is H or R⁴ and R⁵ taken together are —CH₂— as part of a cyclopropa group; [symbol]
is independently in each case either a single bond or a double bond but is a single bond when part of a cyclopropa group;
or a pharmaceutically acceptable salt, ester or ether thereof.

2. A compound or the pharmaceutically acceptable salt, ester or ether thereof according to claim 1 which is a compound of the formula III 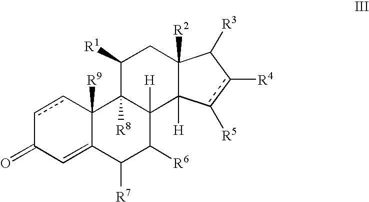
wherein:
R¹ is —OH or ═O;
R² is (C₁₋₃)alkyl or (C₂₋₃)alkenyl;
R³ is selected from: 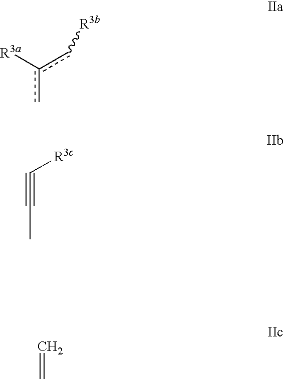
Wherein the lowermost carbon is carbon 17 of the D ring
and wherein:
R^{3a} is H, halogen, monocyclic aryl or is (C₁₋₅)alkyl optionally substituted with hydroxy, halogen, (C₁₋₆)alkoxy or (C₁₋₆)acyloxy;
R^{3b} is H, (C₁₋₃)alkyl or halogen; and
R^{3c} is H, (C₁₋₆)alkyl, (C₂₋₆)alkenyl or (C₁-C₆)alkynyl;
R⁴ is H or (C₁₋₆)alkyl;
R⁵ is H or R⁴ and R⁵ taken together are —CH₂— as part of a 15,16-cyclopropa group;
R⁶ is H, —CN, (C₁₋₆)alkyl, carboxyl(C₁₋₄)alkyl, carboxyl, —C(═O)O(C₁₋₄)alkyl (C₁₋₅)alkylthio, or (C₁₋₅)acylthio.
R⁷ is H or halogen, or R⁶ and R⁷ taken together are —CH₂— as part of a 6,7 cyclopropa group or, taken together, R⁶ and R⁷ form the second bond of a double bond;
R⁸ is H or a halogen atom, or, taken together, R¹ and R⁸ form the second bond of a double bond;
R⁹ is H or (C₁₋₄alkyl; and [symbol]
is in each case, independently, either a single bond or a double bond but is a single bond when part of a cyclopropa group;
or a pharmaceutically acceptable ester or ether thereof.

3. A compound or the pharmaceutically acceptable salt, ester or ether thereof, according to claim 1 in which R¹ is —OH.

4. A compound or the pharmaceutically acceptable salt, ester or ether thereof according to claim 1 in which R² is methyl or ethyl.

5. A compound or the pharmaceutically acceptable salt, ester or ether thereof according to claim 1 in which R^{3a} is methyl or ethyl optionally substituted with halogen, methoxy or (C₁₋₃)acyloxy.

6. A compound or the pharmaceutically acceptable salt, ester or ether thereof according to claim 1 in which R^{3a} is methyl or ethyl.

7. A compound or the pharmaceutically acceptable salt, ester or ether thereof according to claim 1 in which R^{3b} is H or methyl.

8. A compound or the pharmaceutically acceptable salt, ester or ether thereof according to claim 1 in which R^{3c} is H.

9. A compound or the pharmaceutically acceptable salt, ester or ether thereof according to claim 1 in which R³ is of the formula IIa.

10. A compound or the pharmaceutically acceptable salt, ester or ether thereof according to claim 1 in which R⁴ is methyl or ethyl.

11. A compound or the pharmaceutically acceptable salt, ester or ether thereof according to claim 1 in which R⁶ is H, —CN, (C₁₋₄)alkyl, carboxyl, —C(═O)OCH₃, (C₁₋₅)acylthio.

12. A compound or the pharmaceutically acceptable salt, ester or ether thereof according to claim 1 in which R⁶ is H, methyl, ethyl, propyl, carboxyl, —C(═O)OCH₃ or —S(C═O)CH₃.

13. A compound or the pharmaceutically acceptable salt, ester or ether thereof according to claim 1 in which R⁶ is H, methyl or —S(C═O)CH₃.

14. A compound or the pharmaceutically acceptable salt, ester or ether thereof according to claim 1 in which R⁹ is methyl.

15. A compound of the formula I having a steroid skeleton and substitution characteristics in the A and B rings of the steroid skeleton effective for mineralocorticoid receptor antagonism, and rings C and D of the steroid skeleton having substituents thereon according to formula I 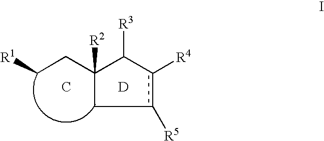
wherein:
R¹ is —OH or ═O;
R² is (C₁₋₃)alkyl or (C₂₋₃)alkenyl;
R³ is selected from: 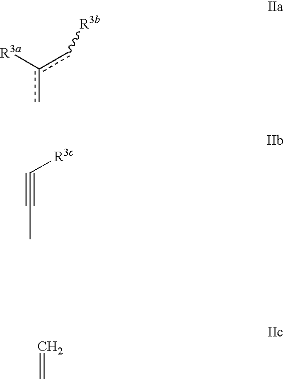
Wherein the lowermost carbon is carbon 17 of the D ring
R^{3a} is H, halogen, monocyclic aryl or is (C₁₋₅)alkyl optionally substituted with hydroxy, halogen, (C₁₋₆)alkoxy or (C₁₋₆)acyloxy;
R^{3b} is H, (C₁₋₃)alkyl or halogen; and
R^{3c} is H, (C₁₋₆)alkyl, (C₂₋₆)alkenyl or (C₁-C₆)alkynyl;
R⁴ is H or (C₁₋₆)alkyl;
R⁵ is H or R⁴ and R⁵ taken together are —CH₂— as part of a 15,16-cyclopropa group;
or a pharmaceutically acceptable salt, ester or ether thereof,
with the proviso that (11β)-11-hydroxy-pregn-4-en-3-one, (11β-20S)-11,21-dihydroxy-20-methylpregn-4-en-3-one and (11β-20S)-11,21-dihydroxy-20-methyl-pregn-1,4-dien-3-one are excluded.

16. A compound of the formula I or the pharmaceutically acceptable salt, ester or ether thereof according to claim 15 which is a compound of the formula III 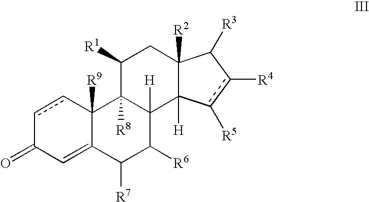
wherein:
R¹ is —OH or ═O;
R² is (C₁₋₃)alkyl or (C₂₋₃)alkenyl;
R³ is selected from: 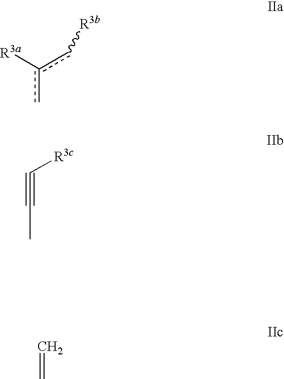
Wherein the lowermost carbon is carbon 17 of the D ring and wherein:
R^{3a} is H, halogen, monocyclic aryl or is (C₁₋₅)alkyl optionally substituted with hydroxy, halogen, (C₁₋₆)alkoxy or (C₁₋₆)acyloxy;
R^{3b} is H, (C₁₋₃)alkyl or halogen; and
R^{3c} is H, (C₁₋₆)alkyl, (C₂₋₆)alkenyl or (C₁₋₆)alkynyl;
R⁴ is H or (C₁₋₆)alkyl;
R⁵ is H or R⁴ and R⁵ taken together are —CH₂— as part of a 15,16-cyclopropa group;
R⁶ is H, —CN, (C₁₋₆)alkyl, carboxyl(C₁₋₄)alkyl, carboxyl, —C(═O)O(C₁₋₄)alkyl (C₁₋₅)alkylthio, or (C₁₋₅)acylthio;
R⁷ is H or halogen, or R⁶ and R⁷ taken together are —CH₂— as part of a 6,7-cyclopropa group or, taken together, R⁶ and R⁷ form the second bond of a double bond;
R⁸ is H or a halogen atom, or, taken together, R¹ and R⁸ form the second bond of a double bond;
R⁹ is H or (C₁₋₄)alkyl; and [symbol]
is in each case, independently, either a single bond or a double bond but is a single bond when part of a cyclopropa group;
or a pharmaceutically acceptable ester or ether thereof,
with the proviso that (11β)-11-hydroxy-pregn-4-en-3-one, (11β-20S)-11,21-dihydroxy-20-methylpregn-4-en-3-one and (11β-20S)-11,21-dihydroxy-20-methyl-pregn-1,4-dien-3-one are excluded.

17. (canceled)

18. A pharmaceutical composition comprising a compound of the formula I according to claim 1 or a pharmaceutically acceptable salt, ester or ether thereof.

19. A method of treatment of a condition associated with the mineralocorticoid receptor, comprising administering to a patient in need thereof, a pharmaceutically effective amount of a compound of formula I according to claim 14, or a pharmaceutically acceptable salt, ester or ether thereof.

20. A method of treatment of a condition associated with the mineralocorticoid receptor, comprising administering to a patient in need thereof, a pharmaceutically effective amount of a compound of formula I according to claim 1, or a pharmaceutically acceptable salt, ester or ether thereof.

21. A pharmaceutical composition comprising a compound of the formula I according to claim 14 or a pharmaceutically acceptable salt, ester or ether thereof.
